(19) United States
(12) Patent Application Publication
(10) Pub. No.: US 2021/0121454 A1
(43) Pub. Date: Apr. 29, 2021

(54) METHOD OF TREATING LAMINOPATHIES

(71) Applicant: Eiger BioPharmaceuticals, Inc., Palo Alto, CA (US)

(72) Inventors: Richard Anthony Franco, JR., Raleigh, NC (US); Colin Michael Hislop, Menlo Park, CA (US)

(21) Appl. No.: 16/665,899

(22) Filed: Oct. 28, 2019

(51) Int. Cl. A61K 31/4545; A61K 31/451; A61K 31/5517; A61K 31/4439

(52) U.S. Cl. CPC A61K 31/4545; A61K 31/4439; A61K 31/5517; A61K 31/451

(57) ABSTRACT

Methods of treating progeria, including HGPS and PL, are provided. In some embodiments, the method comprises administering to a subject having progeria a formulation of lonafarnib.

### FIELD

This disclosure relates to treatment of laminopathies, including Hutchinson-Gilford Progeria Syndrome (HGPS) and Progeroid laminopathies (PL), cellular aging and aging-related conditions, and more particularly to the use of lonafarnib and compositions to treat such conditions.

### BACKGROUND

HGPS is caused by a mutation in exon 11 of the lamin A/C (LMNA) gene, which codes for the nuclear lamina proteins, lamin A and lamin C, proteins necessary for maintaining the integrity of the nuclear membranes (Eriksson et al., Nature. 2003 May 15; 423(6937):293-8; Mounkes et al., Nature. 2003 May 15; 423(6937):298-301). The primary ribonucleic acid (RNA) transcript of the LMNA gene contains 12 exons. Alternative splicing of RNA encoded by exon 10 generates lamin C and a lamin A precursor, prelamin A. Prelamin A must be post-translationally processed outside the nucleus via farnesylation, cleavage of the last three amino acids at its carboxy terminal CAAX residues, and methyl esterification. Inside the nucleus, prelamin A undergoes proteolysis of its C-terminal 15 amino acids to become mature lamin A.

Lamin A expression is developmentally regulated and is expressed in most somatic cells. The multisystemic disease processes in HGPS are downstream of this abnormal protein defect. Most (90%) subjects with HGPS have a single cytosine to thymine transition at nucleotide 1824 that does not change the translated amino acid (Gly608Gly), but activates a cryptic splice site, resulting in the deletion of 150 base pairs in the 3′-portion of exon 11. This altered mRNA produces a shortened abnormal protein with a 50-amino acid deletion near its C-terminal end, henceforth called “progerin.” This 50-amino acid deletion does not affect the ability of progerin to localize to the nucleus or to dimerize because the necessary components for these functions are not deleted. Importantly, however, it does remove the recognition site that leads to proteolytic cleavage of the terminal 15 amino acids of prelamin A, along with the phosphorylation site(s) involved in the dissociation and re-association with the nuclear membrane at each cell division. Thus, unlike lamin A, progerin retains the farnesyl group which enables its ability to associate with the inner nuclear membrane and cause cellular damage via structural instability and functional abnormalities that lead to disease (Goldman et al., Proc Natl Acad Sci USA. 2004 Jun. 15; 101(24):8963-8; Cao et al., Proc Natl Acad Sci USA. 2007 Mar. 20; 104(12):4949-54). This accumulation of progerin, specifically farnesylated progerin, is thought to be responsible for the clinical manifestations of the disease (Kieran et al., Pediatrics. 2007 October; 120(4):834-41).

Progeroid laminopathies (PL) have clinical features that overlap with HGPS and are caused by mutations in the LM NA gene or proteins affecting the post-translational pathway of prelamin A such as the metalloproteinase zinc metalloproteinase STE24 (ZMPSTE24) that result in progerin-like proteins (Gordon et al., Bone. 2019 August; 125:103-111).

Children with PL often experience normal fetal and early post-natal development, but present with a wide range of heterogeneous phenotypes usually within the first year of life. Clinical features may not become evident until later in the first decade of life (Starke et al., Aging (Albany N.Y.). 2013 Jun.; 5(6):445-59). Most children with HGPS die from cardiovascular disease in their second decade (Meredith et al., 2008). Due to the acceleration of cardiovascular disease, death in individuals with HGPS occurs almost exclusively from myocardial infarction, heart failure, or stroke, which are sequelae of widespread arteriosclerosis, while in PL, these are common causes of death. Myocardial infarction caused death in approximately 90% of individuals with HGPS, with stroke causing death in the remaining 10% (Ullrich & Gordon, Handbook Clin Neurol. 2015; 132(3rd series), Neurocutaneous Syndromes, Chapter 18, pages 249-264 “Ullrich & Gordon, 2015”)). The average lifespan of subjects with HGPS is approximately 14.5 years (Gordon et al., Circulation. 2014 Jul. 1; 130(1):27-34; Ullrich & Gordon, 2015; Gordon et al., PRF By The Numbers. Slideshow, www.progeriaresearch.org. The Progeria Research Foundation, Inc., 68 pages, May 2018).

There is a need in the art to understand the safety and toxicity, methods of using lonafarnib, and methods to reduce the adverse events associated with the use of lonafarnib due to the anticipated long duration treatment in a pediatric population.

### SUMMARY

Disclosed herein are methods of treating laminopathies, including cellular aging and cellular aging-related conditions (e.g., cardiovascular diseases or conditions), which result from expression of progerin, a mutant lamin A protein, or an abnormal lamin A protein such as overabundance of prelamin A. In particular, the disclosure provides methods of treating laminopathies (e.g., HGPS or PL) with lonafarnib at doses that are tolerable for long term treatment (e.g., between 12 months to 25 years or more). According to one aspect, provided herein are methods of treating subjects with laminopathies comprising administering about 150 mg/m² lonafarnib to the subjects. According to another aspect, the disclosure features a method of treating a subject with laminopathies comprising administering 150 mg/m² rounded to the nearest 25 mg/m² of lonafarnib to a subject, wherein the subject is further administered loperamide at a daily dose not to exceed 1 mg.

According to some embodiments of the aspects and embodiments herein, the subject is administered about 100 mg/m² to about 200 mg/m² lonafarnib. According to some embodiments of the aspects and embodiments herein, the subject is administered about 100 mg/m² to about 175 mg/m² lonafarnib. According to some embodiments of the aspects and embodiments herein, the subject is administered about 100 mg/m² to about 150 mg/m² lonafarnib. According to some embodiments of the aspects and embodiments herein, the subject is administered about 100 mg/m² to about 120 mg/m² lonafarnib. According to some embodiments of the aspects and embodiments herein, the subject is administered about 125 mg/m² to about 200 mg/m² lonafarnib. According to some embodiments of the aspects and embodiments herein, the subject is administered about 125 mg/m² to about 175 mg/m² lonafarnib. According to some embodiments of the aspects and embodiments herein, the subject is administered about 125 mg/m² to about 150 mg/m² lonafarnib. According to some embodiments of the aspects and embodiments herein, the subject is administered about 150 mg/m² to about 200 mg/m² lonafarnib. According to some embodiments of the aspects and embodiments herein, the subject is administered about 150 mg/m² to about 175 mg/m² lonafarnib. According to some embodiments of the aspects and embodiments herein, the subject is administered about 175 mg/m² to about 200 mg/m² lonafarnib. According to some embodiments of the aspects and embodiments herein, the lonafarnib is administered about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m². According to some embodiments of the aspects and embodiments herein, the amount of lonafarnib administered is rounded to the nearest 25 mg or 25 mg/m² of lonafarnib to subjects. According to one embodiment, provided herein are methods of treating subjects with laminopathies comprising administering about 150 mg/m²′ rounded to the nearest 25 mg or 25 mg/m² of lonafarnib to subjects. According to some embodiments of the aspects and embodiments herein, a subject is administered lonafarnib for from between 12 months and 25 years. According to some embodiments of the aspects and embodiments herein, a subject is administered lonafarnib for between one to 25 years or more. According to some embodiments of the aspects and embodiments herein, a subject is administered lonafarnib for about 1, 2, 3, 4, 5, 6, 7, 8, 9, 10, 11, 12, 13, 14, 15, 16, 17, 18, 19, 20, 21, 22, 23, 24, 25 or more years. According to some embodiments of the aspects and embodiments herein, a subject is administered lonafarnib beginning at the age of 12 months. According to some embodiments of the aspects and embodiments herein, the subject is further administered loperamide at a daily dose not to exceed 1 mg. In one embodiment, increases in dose of loperamide are made gradually. According to some embodiments of the aspects and embodiments herein, the dose is increased by 0.1 to about 0.2 mg. According to some embodiments of the aspects and embodiments herein, lonafarnib is administered without food. According to some embodiments of the aspects and embodiments herein, lonafarnib is administered with food. According to some embodiments of the aspects and embodiments herein, lonafarnib increases loperamide Cₘₐₓ by about 3-fold compared to loperamide administered alone. According to some embodiments of the aspects and embodiments herein, lonafarnib increases loperamide Cₘₐₓ by about 1.5-fold compared to loperamide administered alone.

According to some embodiments of the aspects and embodiments herein, lonafarnib increases loperamide Cₘₐₓ by about 2-fold compared to loperamide administered alone. According to some embodiments of the aspects and embodiments herein, lonafarnib increases loperamide Cₘₐₓ by about 2.5-fold compared to loperamide administered alone. According to some embodiments of the aspects and embodiments herein, lonafarnib increases loperamide Cₘₐₓ by about 3.5-fold compared to loperamide administered alone. According to some embodiments of the aspects and embodiments herein, lonafarnib increases loperamide Cₘₐₓ by about 4-fold or more compared to loperamide administered alone. According to some embodiments of the aspects and embodiments herein, lonafarnib increases loperamide AUC₀₋ₜ by about 4-fold compared to loperamide administered alone. According to some embodiments of the aspects and embodiments herein, lonafarnib increases loperamide AUC₀₋ₜ by about 1.5-fold compared to loperamide administered alone. According to some embodiments of the aspects and embodiments herein, lonafarnib increases loperamide AUC₀₋ₜ by about 2-fold compared to loperamide administered alone. According to some embodiments of the aspects and embodiments herein, lonafarnib increases loperamide AUC₀₋ₜ by about 2.5-fold compared to loperamide administered alone. According to some embodiments of the aspects and embodiments herein, lonafarnib increases loperamide AUC₀₋ₜ by about 3-fold compared to loperamide administered alone. In one embodiment, lonafarnib increases loperamide AUC₀₋ₜ by about 3.5-fold compared to loperamide administered alone. According to some embodiments of the aspects and embodiments herein, lonafarnib increases loperamide AUC₀₋ₜ by about 4.5-fold compared to loperamide administered alone. According to some embodiments of the aspects and embodiments herein, lonafarnib increases loperamide AUC₀₋ₜ by about 5-fold or more compared to loperamide administered alone. According to some embodiments of the aspects and embodiments herein, sensitive substrates of CYP3A are contraindicated in subjects being administered lonafarnib.

According to another aspect, the disclosure provides a method of treating a subject with laminopathies comprising administering 150 mg/m² rounded to the nearest 25 mg/m² of lonafarnib to a subject, wherein the sensitive substrates of CYP3A are contraindicated in subjects being administered lonafarnib. According to some embodiments, the subject is further administered midazolam. According to some embodiments, the subject is further administered midazolam and wherein there is about a 0.5-hour delay in Tₘₐₓ of midazolam when co-administered with lonafarnib. According to some embodiments, the increase in midazolam's systemic exposure are via a mechanism-based inhibition of cytochrome P450 CYP3A.

According to another aspect, the disclosure provides a method of treating a subject with laminopathies comprising administering 150 mg/m² rounded to the nearest 25 mg/m² of lonafarnib to a subject, wherein lonafarnib is administered with food. According to some embodiments, the food is a high fat meal. According to some embodiments, the food it a low-fat, low-calorie meal. In one embodiment, food decreases lonafarnib Cₘₐₓ from between 25% to about 55% as compared to a fasted state. According to some embodiments, the food decreases lonafarnib Cₘₐₓ about 25%, about 30%, about 35%, about 40%, about 45, about 50%, about 55% or more. According to some embodiments, the food decreases lonafarnib exposure (AUC₀₋ₜ and AUC_{0-inf}) from about 20% to about 60%, or from about 25% to about 60%, or from about 30% to about 60%, or from about 35% to about 60%, or from about 40% to about 60%, or from about 45% to about 60%, or from about 50% to about 60%, or from about 55% to about 60%, or from about 20% to about 55%, or from about 25% to about 55%, or from about 30% to about 55%, or from about 35% to about 55%, or from about 40% to about 55%, or from about 45% to about 55%, or from about 50% to about 55%, or from about 20% to about 50%, or from about 25% to about 50%, or from about 30% to about 50%, or from about 35% to about 50%, or from about 40% to about 50%, or from about 45% to about 50%, or from about 20% to about 45%, or from about 25% to about 45%, or from about 30% to about 45%, or from about 35% to about 45%, or from about 40% to about 45%, or from about 20% to about 40%, or from about 25% to about 40%, or from about 30% to about 40%, or from about 35% to about 40%, or from about 20% to about 35%, or from about 25% to about 35%, or from about 30% to about 35%, or from about 20% to about 30%, or from about 25% to about 30%, or from about 20% to about 25% compared to a fasted state. According to some embodiments, food decreases lonafarnib exposure (AUC₀₋ₜ and AUC_{0-inf}) from about 10% to about 40%, or from about 10% to about 35%, or from about 10% to about 30%, or from about 10% to about 25%, or from about 10% to about 20%, or from about 10% to about 15%, or from about 15% to about 40%, or from about 15% to about 35%, or from about 15% to about 30%, or from about 15% to about 25%, or from about 15% to about 20%, or from about 20% to about 40%, or from about 20% to about 35%, or from about 20% to about 30%, or from about 20% to about 25%, compared to a fast stated. In one embodiment, food decreases lonafarnib exposure (AUC₀₋ₜ and AUC_{0-inf}) from about 21% to about 29%, for example about 21%, 22%, 23%, 24%, 25%, 26%, 27%, 28%, or 29%. According to some embodiments, the decrease in lonafarnib exposure (AUC₀₋ₜ and AUC_{0-inf}) is compared to a fasted state. According to some embodiments, oral clearance (CL/F) is between about 25% to about 40% higher, about 25% to about 35% higher, about 25% to about 30% higher, about 25%, 26%, 27%, 28%, 29%, 30%, 31%, 32%, 33%, 34%, 35%, 36%, 37%, 38%, 39% or 40% higher when lonafarnib is administered with food as compared to a fasted state. According to some embodiments, oral clearance (CL/F) is between about 33% to about 35% higher as compared to a fasted state when lonafarnib is administered with food. According to some embodiments, the Tₘₐₓ is delayed as compared to a fasted state. According to some embodiments, the subject is further administered omeprazole and wherein omeprazole Cₘₐₓ is increased following coadministration with lonafarnib. According to some embodiments, the increase in omeprazole Cₘₐₓ is between about 30% and about 50% following coadministration with lonafarnib. According to some embodiments, the increase in omeprazole Cₘₐₓ is between about 40% and about 50% following coadministration with lonafarnib. According to some embodiments, the increase in omeprazole Cₘₐₓ is between about 45% and about 50% following coadministration with lonafarnib. According to some embodiments, the subject is further administered omeprazole and wherein omeprazole Cₘₐₓ is increased about 44% following coadministration with lonafarnib. According to some embodiments, one or more of the omeprazole Cₘₐₓ following coadministration with LNF is at about the same as the Tₘₐₓ, or theomeprazole exposures (AUC₀₋ₜ and AUC_{0-inf}) were increased approximately 2-fold when omeprazole was coadministered with lonafarnib. According to some embodiments, omeprazole CL/F and Kₑₗ following coadministration with lonafarnib are decreased and the T_{1/2} is increased. According to some embodiments, CYP2C19 substrates are monitored during concomitant administration with lonafarnib. In one embodiment, the lonafarnib is provided as 50 mg or 75 mg capsules. According to some embodiments, the laminopathies comprise one or more of Hutchinson-Gilford Progeria Syndrome and Progeroid Laminopathies. According to some embodiments, the laminopathies are associated with production of abnormally farnesylated lamin A proteins. According to some embodiments, one or more of the following is contraindicated with the use of lonafarnib: α1-adrenoreceptor antagonist, analgesics, antianginal, antineoplastic, antiarrhythmics, anti-gout, antimycobacterial, antipsychotics, antibiotic, antihistamines, ergot derivatives, GI motility agent, HMG Co-A reductase inhibitor, phosphodiesterase inhibitor, sedatives and hypnotics. According to some embodiments, one or more of the following is contraindicated with the use of lonafarnib alfuzosin, propoxyphene, ranolazine, venetoclax, amiodarone, bepridil, dronedarone, quinidine, colchicine, rifabutin, lurasidone, clozapine, pimozide, quetiapine, fusidic acid, astemizole, terfenadine, dihydroergotamine, ergonovine, ergotamine, methylergonovine, cisapride, lovastatin, simvastatin, avanafil, sildenafil, vardenafil, clorazepate, diazepam, estazolam, flurazepam, midazolam and triazolam. According to some embodiments, concomitant use with strong or moderate CYP3A inhibitors or inducers is contraindicated.

Further aspects and embodiments are disclosed infra.

### DETAILED DESCRIPTION

The terminology used herein is for the purpose of describing particular aspects and embodiments only, and is not intended to be limiting. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods and materials are now described. All technical and patent publications cited herein are incorporated herein by reference in their entirety.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated, that all numerical designations are preceded by the term “about.” References to ranges include all numbers in-between and the end-points unless indicated otherwise.

The singular forms “a,” “an,” and “the” include plural referents unless the context clearly dictates otherwise. Thus, for example, reference to “a compound” includes a plurality of compounds. The term “comprising” is intended to mean that the compounds, compositions and methods include the recited elements, but not excluding others. “Consisting essentially of” when used to define compounds, compositions and methods, means excluding other elements that would materially affect the basic and novel characteristics of the claimed invention. “Consisting of” means excluding any element, step, or ingredient not specified in the claim. Embodiments defined by each of these transition terms are within the scope of this invention.

The term “formulation” or “pharmaceutical formulation,” as used herein, refers to a composition suitable for administration to a subject. A pharmaceutical formulation may be sterile, and preferably free of contaminants that are capable of eliciting an undesirable response within the subject (e.g., the compounds in the pharmaceutical formulation are pharmaceutical grade). Pharmaceutical formulations can be designed for administration to subjects or subjects in need thereof via a number of different routes of administration, including oral, intravenous, buccal, rectal, parenteral, intraperitoneal, intradermal, intramuscular, subcutaneous, inhalational and the like. In some embodiments, a pharmaceutical formulation as described herein is formulated for oral administration.

As used herein, a “therapeutically effective amount” is an amount of an active ingredient (e.g., lonafarnib or its pharmaceutically acceptable salt) that eliminates, ameliorates, alleviates, or provides relief of the symptoms or leads to clinical outcomes for which it is administered.

The terms “treatment,” “treating,” and “treat,” as used herein in reference to administering lonafarnib to treat HGPS or PL (together sometimes referred to as “progeria” or “laminopathies”), and covers any treatment of the disease in a human subject, and includes reducing the risk, frequency or severity of progeria, impeding the development of the disease; and/or relieving the disease, i.e., causing regression of the disease and/or relieving one or more disease symptoms.

The terms “administer,” “administering,” and “administration” as used herein, refer to introducing a compound (e.g., avexitide), a composition, or an agent into a subject or patient, such as a human. As used herein, the terms encompass both direct administration, (e.g., self-administration or administration to a patient by a medical professional) and indirect administration (e.g., the act of prescribing a compound or composition to a subject).

“QD” and “BID” have their usual meanings of administration of a composition once per day or twice per day, respectively. In some embodiments, administration once per day (QD) means that at least 20 hours, at least 22 hours, or about 24 hours elapse between administrations. In some embodiments, administration once per day means administration about every 24 hours. In some embodiments, administration twice per day (BID) means that at least 4 hours, at least 6 hours, at least 8 hours, at least 10 hours, at least 11 hours, or about 12 hours elapse between administrations. In some embodiments, administration twice per day means administration about every 12 hours.

As used herein, the terms “patient” and “subject” interchangeably refer to an individual (e.g., a human or a non-human mammal). In some embodiments the subject has laminopathies, which in include HGPS and PL.

In one aspect the methods described herein include treating a subject with laminopathies by administering 150 mg/m² rounded to the nearest 25 mg/m² of lonafarnib to the subject. In some embodiments, the subject is administered lonafarnib for from between 12 months and 25 years. In some embodiments, the subject is administered lonafarnib beginning at the age of 12 months. In some embodiments, the subject is further administered loperamide at a daily dose not to exceed 1 mg. In some embodiments, the dose may be increased above 1 mg, but increases are to be made gradually. According to some embodiments, the increases in dose of loperamide occur weekly. According to some embodiments, the increases in dose of loperamide occur daily. Gradually, as used herein, includes increasing the loperamide dose by about 0.1 mg/week; by about 0.2 mg/week, by about 0.05 mg/week, by about 0.1 mg/day, by about 0.2 mg/day, or by about 0.05 mg/day. The subject should be monitored closely while increasing the dose and the increase in dose should not continue once diarrhea symptoms are tolerable by the subject or caregiver.

In some embodiments, lonafarnib increases loperamide Cₘₐₓ by about 3-fold compared to loperamide administered alone. In some embodiments, lonafarnib increases loperamide AUC₀₋ₜ by about 4-fold compared to loperamide administered alone. In some embodiments, sensitive substrates of CYP3A are contraindicated in subjects being administered lonafarnib.

In some embodiments, sensitive substrates of CYP3A comprises one or more of benzodiazepines, budesonide, dronedaroe, sildenafil, ticagrelor or other sensitive substrates of CYP3A known to those of skill in the art.

In some embodiments, the benzodiazepine comprises midazolam. In some embodiments, lonafarnib results in a 0.5-hour delay in Tₘₐₓ of midazolam when co-administered. In some embodiments, the significant increase in midazolam's systemic exposure are via a mechanism-based inhibition of cytochrome P450 CYP3A.

In some embodiments, lonafarnib is administered with food. In some embodiments, food is a high fat meal or a low-fat, low-calorie meal. In some embodiments, food decreases lonafarnib Cₘₐₓ from between 25% to about 55% as compared to a fasted state.

In some embodiments, food decreases lonafarnib exposure (AUC₀₋ₜ and AUC_{0-inf}) from about 21% to about 29%. In some embodiments, oral clearance (CL/F) is between about 33% to about 35% higher as compared to a fasted state when lonafarnib is administered with food. In some embodiments, the Tₘₐₓ is delayed as compared to a fasted state. It is recommended that lonafarnib, despite a significant decrease in exposure, be administered with food. This is to alleviate certain adverse events, e.g., diarrhea, nausea, abdominal pain, and vomiting. The significant decrease in lonafarnib exposure with food was an unexpected and surprising finding.

In some embodiments, omeprazole Cₘₐₓ was increased 44% following coadministration with multiple-dose lonafarnib and was observed at approximately the same time (Tₘₐₓ) In some embodiments, omeprazole exposures (AUC₀₋ₜ and AUC_{0-inf}) were increased approximately 2-fold when omeprazole was coadministered with lonafarnib. In some embodiments, omeprazole CL/F and Kₑₗ following coadministration with lonafarnib, were both decreased, while T_{1/2} was increased. In some embodiments, CYP2C19 substrates should be monitored during concomitant administration with lonafarnib.

In some embodiments, the laminopathies comprise one or more of Hutchinson-Gilford Progeria Syndrome and Progeroid Laminopathies

In some embodiments, the laminopathies are associated with production of abnormally farnesylated lamin A proteins (e.g., ZMPSTE24 mutations that cause Mandibuloacral dysplasia type B) in subjects 12 months of age and older.

In some embodiments, the lonafarnib is provided as 50 mg or 75 mg capsules. The recommended dosage regimen in subjects 12 months of age and older with a confirmed diagnosis of Hutchinson-Gilford Progeria Syndrome (HGPS) or a Progeroid Laminopathy (PL) associated with the production of abnormally farnesylated lamin A proteins is 150 mg/m² twice daily with the morning and evening meals, approximately 12 hours apart (not to exceed 400 mg daily). All dosages should be rounded to nearest 25 mg increment.

For subjects experiencing unmanageable adverse drug reactions, the dose of lonafarnib can be reduced to 115 mg/m² per dose. According to some embodiments, all dosages should be rounded to the nearest 25 mg increment.

TABLE 1
BSA-based Dosing for the Recommended Dose of 150 mg/m²

| | | | | Morning | | Evening | |
|---|---|---|---|---|---|---|---|
| | | | | (AM) Dosing | | (PM) Dosing | |
| | | | Rounded | 50 mg | 75 mg | 50 mg | 75 mg |
| | | Total Daily | to Nearest | (yellow | (light orange | (yellow | (light orange |
| BSA (m2) | Dose (mg) | Dose (mg) | 25 mg | capsule) | capsule) | capsule) | capsule) |
| 0.3 | 45 | 90 | 100 | 1 | | 1 | |
| 0.4 | 60 | 120 | 125 | | 1 | 1 | |
| 0.5 | 75 | 150 | 150 | | 1 | | 1 |
| 0.6 | 90 | 180 | 175 | 2 | | | 1 |
| 0.7 | 105 | 210 | 200 | 2 | | 2 | |
| 0.8 | 120 | 240 | 250 | 1 | 1 | 1 | 1 |
| 0.9 | 135 | 270 | 275 | | 2 | 1 | 1 |
| 1 | 150 | 300 | 300 | | 2 | | 2 |
| 1.1 | 165 | 330 | 325 | 2 | 1 | | 2 |
| 1.2 | 180 | 360 | 350 | 2 | 1 | 2 | 1 |
| 1.3 | 195 | 390 | 400 | 4 | | 4 | |

BSA-based Dosing for a Dose of 115 mg/m²

| | | | | Morning | | Evening | |
|---|---|---|---|---|---|---|---|
| | | | | (AM) Dosing | | (PM) Dosing | |
| | | | Rounded | 50 mg | 75 mg | 50 mg | 75 mg |
| | | Total Daily | to Nearest | (yellow | (light orange | (yellow | (light orange |
| BSA (m2) | Dose (mg) | Dose (mg) | 25 mg | capsule) | capsule) | capsule) | capsule) |
| 0.3 | 0 | 0 | 75 | | 1 | | |
| 0.4 | 0 | 0 | 100 | 1 | | 1 | |
| 0.5 | 0 | 0 | 125 | | 1 | 1 | |
| 0.6 | 0 | 0 | 150 | | 1 | | 1 |
| 0.7 | 0 | 0 | 150 | 2 | | 2 | |
| 0.8 | 0 | 0 | 175 | 2 | | | 1 |
| 0.9 | 0 | 0 | 200 | 2 | | 2 | |
| 1 | 0 | 0 | 225 | 1 | 1 | 2 | |
| 1.1 | 0 | 0 | 250 | 1 | 1 | 1 | 1 |
| 1.2 | 0 | 0 | 275 | | 2 | 1 | 1 |
| 1.3 | 0 | 0 | 300 | | 2 | | 2 |
| 1.4 | 0 | 0 | 325 | 2 | 1 | | 2 |
| 1.5 | 0 | 0 | 350 | 2 | 1 | 2 | 1 |
| 1.6 | 0 | 0 | 375 | 1 | 2 | 2 | 1 |
| 1.7 | 0 | 0 | 400 | 4 | | 4 | |

A survival benefit has been observed with this treatment and chronic therapy is recommended.

Missed Dose

If a dose is missed, subjects should take the dose as soon as possible and any time up to 8 hours prior to the next scheduled dose (12 hours±4 hours). All doses should be taken with food. If less than 8 hours remains before the next scheduled dose, subjects should not take the missed dose, and resume taking lonafarnib at the next scheduled dose.

Renal Impairment

Lonafarnib is renally cleared <1% and no meaningful differences would be expected in subjects with renal impairment. However, results of a study demonstrate that lonafarnib exposures increase in subjects with severe renal impairment. Thus, lonafarnib is contraindicated in in subjects with severe renal impairment.

Hepatic Impairment

Lonafarnib is extensively metabolised via the cytochrome P450 enzyme system in the liver. Lonafarnib has not been studied in subjects with hepatic impairment. Study results from single-dose lonafarnib in combination with ritonavir in mild and moderate hepatically impaired subjects showed similar lonafarnib exposures relative to the matched normal control group (normal hepatic function). These results indicate no dose adjustments are warranted for lonafarnib in subjects with mild or moderate hepatic impairment.

### Method of Administration

According to some embodiments, lonafarnib capsules are administered orally. According to some embodiments, lonafarnib capsules are taken with food and swallowed whole with a sufficient amount of water. According to some embodiments, capsules should not be chewed.

For subjects unable to swallow capsules, the contents of lonafarnib can be mixed with Ora Blend SF® or Ora-Plus®. For subjects unable to tolerate Ora Blend SF® or Ora-Plus®, the contents of lonafarnib capsules can be mixed with orange juice or applesauce. Do not mix with grapefruit juice or any juice that specifically contains Seville oranges. Pour all contents of lonafarnib onto soft food (applesauce) or mix with a small amount of liquid (Ora Blend SF®, Ora-Plus®, orange juice) at concentrations of 2.5 to 25 mg/mL, mix thoroughly with a spoon and consume entire serving. The mixture must be prepared fresh for each dose and be taken within approximately 10 minutes of mixing.

Lonafarnib should be Taken with Food

In vitro and in vivo studies have demonstrated that lonafarnib is a potent CYP3A time-dependent and mechanism based inhibitor. The following medicines are contraindicated when used with lonafarnib and unless otherwise noted, the contraindication is based on the potential for lonafarnib to inhibit metabolism of the co-administered medicinal product, resulting in increased exposure to the co-administered medicinal product and risk of clinically significant adverse effects.

TABLE 3
Exposure changes when co-administered with lonafarnib

| | | |
|---|---|---|
| Medicinal Product | Medicinal Product | |
| Class | within Class | Rationale |

Concomitant medicinal product levels increased or decreased

| | | |
|---|---|---|
| α₁-Adrenoreceptor | Alfuzosin | Increased plasma concentrations of alfuzosin which |
| Antagonist | | may lead to severe hypotension |
| Analgesics | Propoxyphene | Increased plasma concentrations of propoxyphene |
| | | thereby increasing the risk of severe respiratory |
| | | depression |
| Antianginal | Ranolazine | Increased plasma concentrations of ranolazine which |
| | | may increase the potential for serious and/or life- |
| | | threatening reactions |
| Antineoplastic | Venetoclax | Increased plasma concentrations of venetoclax which |
| | | may lead to increased risk of tumour lysis syndrome |
| | | at the dose initiation and during the dose-titration |
| | | phase (see venetoclax SmPC) |
| Antiarrhythmics | Amiodarone, bepridil, | Increased plasma concentrations of amiodarone, |
| | dronedarone, | bepridil, dronedarone, quinidine. Thereby, increasing |
| | quinidine | the risk of arrhythmias or other serious adverse |
| | | effects from these agents |
| Anti-gout | Colchicine | Increased plasma concentrations of colchicine which |
| | | may lead to serious and/or life-threatening reactions |
| | | in subjects with renal and/or hepatic impairment |
| Antimycobacterial | Rifabutin | Increased plasma concentrations of rifabutin which |
| | | may increase risk of adverse reactions including |
| | | uveitis |
| Antipsychotics | Lurasidone | Increased plasma concentrations of lurasidone which |
| | | may increase the potential for serious and/or life- |
| | | threatening reactions |
| | Clozapine, pimozide | Increased plasma concentrations of clozapine and |
| | | pimozide. Thereby, increasing the risk of serious |
| | | haematologic abnormalities, or other serious adverse |
| | | effects from these agents |
| | Quetiapine | Increased plasma concentrations of quetiapine which |
| | | may lead to coma |
| Antibiotic | Fusidic acid | Increased plasma concentrations of fusidic acid and |
| | | lonafarnib |
| Antihistamines | Astemizole, | Increased plasma concentrations of astemizole and |
| | terfenadine | terfenadine. Thereby, increasing the risk of serious |
| | | arrhythmias from these agents |
| Ergot derivatives | Dihydroergotamine, | Increased plasma concentrations of ergot derivatives |
| | ergonovine, | leading to acute ergot toxicity, including vasospasm |
| | ergotamine, | and ischaemia |
| | methylergonovine | |
| GI motility agent | Cisapride | Increased plasma concentrations of cisapride. |
| | | Thereby, increasing the risk of serious arrhythmias |
| | | from this agent |
| HMG Co-A | Lovastatin, simvastatin | Increased plasma concentrations of lovastatin and |
| reductase inhibitor | | simvastatin; thereby, increasing the risk of myopathy |
| | | including rhabdomyolysis |
| Phosphodiesterase | Avanafil, sildenafil, | Increased plasma concentrations of avanafil, |
| inhibitor | vardenafil | sildenafil, vardenafil, increasing risk of adverse effects |
| Sedatives/hypnotics | Clorazepate, | Increased plasma concentrations of clorazepate, |
| | diazepam, estazolam, | diazepam, estazolam, flurazepam, midazolam and |
| | flurazepam, | triazolam. Thereby, increasing the risk of extreme |
| | midazolam and | sedation and respiratory depression from these |
| | triazolam | agents |

Lonafarnib medicinal product levels increased or decreased

| | | |
|---|---|---|
| Herbal preparation | St. John's wort | Herbal preparations hypericum perforatum due to |
| | | the risk of decreased plasma concentrations and |
| | | reduced clinical effects of lonafarnib. |

Caution should be exercised with concomitant use of lonafarnib and sensitive CYP3A or CYP2C19 substrates and strong or moderate CYP3A inhibitors or inducers. If a subject is taking one of these drugs, the treating physician should consider an alternative drug. If the patient cannot safely discontinue or take an alternative drug, the dose of the inhibitor/inducer should be adjusted per the treating physician and the patient should be monitored for potential adverse effects if taking a drug which is a sensitive CYP3A or CYP2C19 substrate. Concomitant use with certain sensitive CYP3A substrates, strong or moderate CYP3A inhibitors or inducers is contraindicated.

In vitro and in vivo studies have demonstrated that lonafarnib is a potent CYP3A time-dependent and mechanism based inhibitor and moderate CYP2C19 inhibitor. Concomitant administration of lonafarnib with sensitive CYP3A or CYP2C19 substrates and strong or moderate CYP3A4 inhibitors or inducers, including herbal supplements can increase exposure of the co-administered medicinal product or lonafarnib resulting in risk of clinically significant adverse events or decrease exposure of lonafarnib, which may impact efficacy. Medicinal products that are sensitive CYP3A or CYP2C19 substrates, strong or moderate CYP3A inhibitors, or strong or moderate CYP3A inducers should be discontinued. If treatment cannot safety be discontinued or an alternative drug is not available, lonafarnib should be stopped during the course of treatment unless the benefit outweighs the possible risks.

### Effects of Other Medicinal Products on Lonafarnib

Strong or moderate CYP3A inhibitors Lonafarnib is metabolized predominantly via CYP3A4 enzyme. When lonafarnib was co-administered with ketoconazole 200 mg twice daily, a strong CYP3A inhibitor, lonafarnib AUC increased approximately 5-fold and Cₘₐₓ increased approximately 3.6-fold.

The use of the following strong or moderate CYP3A inhibitors is contraindicated with lonafarnib, including, for example, antiarrhythmics (e.g., amiodarone, dronedarone) and antibiotic (e.g., fusidic acid).

Medicinal products that are strong or moderate CYP3A inhibitors should be avoided, and alternatives should be sought, unless the benefit outweighs the possible risks. Example of strong or moderate CYP3A inhibitors including, for example, antibiotics (e.g., chloramphenicol [also CYP2C19 substrate], clarithromycin, isoniazid, telithromycin, troleandomycin, ciprofloxacin, erythromycin); antidepressants (e.g., nefazodone, fluoxetine, fluvoxamine); antiemetic (e.g., aprepitant); antifungals (e.g., itraconazole, ketoconazole, posaconazole, clotrimazole, fluconazole, miconazole, voriconazole); antivirals (e.g., atazanavir, darunavir, indinavir, lopinavir, nelfinavir [also CYP2C19 substrate], ritonavir, saquinavir, tipranavir, amprenavir, dilaverdine, fosamprenavir); calcium channel blockers (e.g., diltiazem, verapamil); H2 receptor antagonist (e.g., cimetidine); other (grapefruit juice, Seville oranges). This list is not intended to be comprehensive and prescribers should check the prescribing information of medicinal products to be co-administered with lonafarnib for potential CYP3A mediated interactions.

Strong or Moderate CYP3A Inducers

Lonafarnib is metabolized predominantly via CYP3A4 enzyme. Medicinal products that induce CYP3A may increase the rate and extent of metabolism of lonafarnib. Co-administration of a strong or moderate CYP3A inducer may reduce the effect of lonafarnib.

The use of the following strong or moderate CYP3A inducers is contraindicated with lonafarnib including, for example, antibiotics (e.g., rifabutin) and other (e.g., St. John's Wort). When co-administering strong or moderate CYP3A inducers with lonafarnib, the possibility of a drug-drug interaction cannot be excluded. Therefore, concomitant use of lonafarnib with a strong or moderate CYP3A inducer should be avoided, and alternatives should be sought. Examples of strong or moderate CYP3A inducers include, for example, antibiotics (e.g., rifampin, nafcillin); anticonvulsants (e.g., carbamazepine, phenytoin, phenobarbital [also CYP2C19 substrate], oxcarbazepine); antifungal (e.g., griseofulvin); antineoplastic (e.g., enzalutamide, mitotane); antivirals (e.g., efavirenz, etravirine); corticosteroid (e.g., dexamethasone); endothelin receptor antagonist (e.g., bosentan); and stimulant (e.g., modafinil). Prescribers should check the prescribing information of medicinal products to be co-administered with lonafarnib for potential CYP3A mediated interactions.

### Effects of Lonafarnib on Other Medicinal Products

Sensitive CYP3A Substrates

Lonafarnib is a strong in vivo CYP3A4 mechanism based inhibitor. When lonafarnib was co-administered with midazolam, a sensitive CYP3A substrate, midazolam AUC increased approximately 7-fold and Cₘₐₓ increased approximately 3-fold.

The use of the following sensitive CYP3A substrates is contraindicated with lonafarnib, including, for example, alpha blocker (alfuzosin); anesthetics/opioids (propoxyphene); antianginal (ranolazine); antineoplastic (venetoclax); antiarrhythmics (bepridil, quinidine); antimycobacterial (rifabutin); anti-gout (colchicine); antipsychotics (lurasidone, clozapine, pimozide, quetiapine); antihistamines (astemizole, terfenadine); anxiolytics/benzodiazepines/sedatives (clorazepate, estazolam, flurazepam, midazolam [including parenterally administered midazolam], triazolam); ergot alkaloid (dihydroergotamine, ergonovine, ergotamine, methylergonovine); GI motility agent (cisapride); lipid lowering (lovastatin, simvastatin); and phosphodiesterase inhibitor (avanafil, sildenafil, vardenafil).

Medicinal products that are sensitive CYP3A substrates should be avoided, and alternatives should be sought, unless the benefit outweighs the possible risks. Patients should be monitored more intensively than usual during this period for potential adverse effects, with dose adjustments made, as necessary. These include, for example, anesthetics/opioids (e.g., alfentanil); antiarrhythmics (e.g., disopyramide, propafenone); antidiabetic (repaglinide); anxiolytics/benzodiazepines/sedatives (e.g., alprazolam, buspirone); immunosuppressive (e.g., cyclosporine, tacrolimus); lipid lowering (e.g., atorvastatin); phosphodiesterase inhibitor (e.g., tadalafil); anticonvulsant (e.g., carbamazepine); antineoplastic (e.g., vinblastine, vincristine, ibrutinib, dasatinib); corticosteroids (e.g., budesonide, dexamethasone, fluticasone, methylprednisolone); anticoagulants (e.g., rivaroxaban, ticagrelor); and vasopressin receptor antagonist (e.g., tolvaptan). Prescribers should check the prescribing information of medicinal products to be co-administered with lonafarnib for potential CYP3A mediated interactions.

Other CYP3A Substrates

In clinical studies, diarrhea was treated with loperamide. Loperamide is metabolized by the cytochrome P450 system via CYP3A and CYP2C8 (major pathways) and CYP2D6 and CYP2B6 (minor pathways). Coadministration of lonafarnib with loperamide increases loperamide exposures up to 4.6-fold. Loperamide dose adjustments are recommended.

Care should be exercised in the co-administration of drugs with a narrow therapeutic index that are metabolized by CYP3A (e.g., paclitaxel, tacrolimus, and cyclosporine).

CYP2C19 Substrates

Lonafarnib is a moderate CYP2C19 inhibitor. When lonafarnib 75 mg twice daily was co-administered with omeprazole, a CYP2C19 substrate, omeprazole AUC increased approximately 1.6-fold and Cmax increased approximately 1.3-fold. Patients taking medicinal products that are CYP2C19 substrates should be monitored during this period for potential adverse effects, with dose adjustments made, as necessary. These include, for example, proton-pump inhibitors (e.g., omeprazole, lansoprazole, pantoprazole, rabeprazole, esomeprazole); antidepressants (e.g., amitriptyline, clomipramine, imipramine, citalopram, escitalopram, moclobemide, bupropion); anticonvulsants (e.g., mephenytoin, nordazepam, phenytoin [also CYP3A inducer], phenobarbital [also CYP3A inducer], primidone, hexobarbital, methylphenobarbital); antineoplastic (e.g., clopidogrel, teniposide); antimalarial (e.g., proguanil); beta blocker (e.g., propranolol); antidiabetic (e.g., gliclazide); muscle relaxant (e.g., carisoprodol); non-steroid anti-inflammatory drugs (e.g., indomethacin); antibiotics (e.g., chloramphenicol [also CYP3A inhibitor]); antiviral (e.g., nelfinavir [also CYP3A inhibitor]); antiandrogen (e.g., nilutamide); steroid hormones (e.g., progesterone); anticoagulants (e.g., warfarin); analgesic (e.g., tapentadol); and other (e.g., limonene). Prescribers should check the prescribing information of medicinal products to be co-administered with lonafarnib for potential CYP2C19 mediated interactions.

P-Gp Substrates

Lonafarnib is a weak P-gp inhibitor. When lonafarnib was co-administered with fexofenadine, a sensitive P gp substrate, fexofenadine AUC and Cₘₐₓ increased approximately 1.2-fold. Patients taking medicinal products that are P-gp substrates with a narrow therapeutic window, such as digoxin, should be carefully monitored.

### EXAMPLES

### Example 1

An open-label, single-center, two-period, single-sequence, multi-drug-drug interaction study to evaluate the effects of multiple-dose lonafarnib on the pharmacokinetics of single-dose midazolam, a sensitive cytochrome p450-3a substrate.

Coadministration of single-dose midazolam with multiple-dose lonafarnib (100 mg twice daily for 5 consecutive days) resulted in large increases in midazolam exposures compared with single-dose midazolam alone.

When multiple-dose lonafarnib was co-administered with single-dose midazolam, the 3 key midazolam PK parameters, Cₘₐₓ, AUC₀₋ₜ, and AUC_{0-inf}, were increased statistically significantly. Coadministration of single dose midazolam with multiple-dose lonafarnib resulted in 0.5-hour delay in Tₘₐₓ; although the Tₘₐₓ delay was statistically significant, and therefore clinically important.

Steady-state lonafarnib (100 mg twice daily) significantly increased midazolam's systemic exposures via mechanism-based inhibition of cytochrome P450 CYP3A. Lonafarnib was found to be a potent in vivo mechanism-based inhibitor (MBI) inhibitor of CYP3A. Given the profound increase, sensitive substrates of CYP3A should be contraindicated with lonafarnib.

In another example, coadministration of single-dose midazolam with multiple-dose lonafarnib resulted in large increases in midazolam exposures; therefore, sensitive substrates of CYP3A should be contraindicated with lonafarnib.

### Example 2

To evaluate the effects of steady-state lonafarnib (LNF) on the pharmacokinetics (PK) of single-dose loperamide in healthy subjects.

The study enrolled 15 healthy subjects. Lonafarnib (100 mg twice daily for 5 consecutive days) was administered; and loperamide (2-mg administered as a single dose on 2 separate occasions) was also administered.

The coadministration of loperamide following multiple-dose lonafarnib increased loperamide exposures as follows: about a 3-fold increase in Cₘₐₓ, about a 4-fold-4.6 fold increase in AUC₀₋ₜ and AUC_{0-inf}, a delay in the Tₘₐₓ by about 2 hours, and loperamide CL/F was decreased 4-fold, all parameters were compared with loperamide administered alone. The Kₑₗ and T_{1/2} were similar. Steady-state was achieved by Day 8, and PK parameters for lonafarnib showed Cₘₐₓ (964 ng/mL) approximately 4 hours after dosing. Extent of exposure was 6,940 ng·h/mL and the T_{1/2} was 4.22 hours.

Dosing of loperamide for the treatment of lonafarnib-induced diarrhea is recommended not to exceed 1 mg taken once daily. If the subject continues to experience clinically significant diarrhea on this dose, the loperamide dose may be increased slowly with caution.

Loperamide is metabolized by the cytochrome P450 system via CYP3A and CYP2C8 (major pathways) and CYP2D6 and CYP2B6 (minor pathways).

Co-administration of lonafarnib with loperamide increases loperamide exposures of loperamide up to 4.6-fold.

The point estimates and corresponding Cis were 3.139 (2.795, 3.526), 3.957 (3.447, 4.536), and 3.987 (3.447, 4.614), for Cₘₐₓ, AUC₀₋ₜ, and AUC_{0-inf}, respectively.

Trough lonafarnib concentrations indicate that steady state was achieved by Day 8.

Coadministration of loperamide with lonafarnib resulted in fewer GI adverse events than when lonafarnib was administered alone, supporting its use for the management of diarrhea, a side effect of lonafarnib.

The results from this study support the continued use of loperamide as an antidiarrheal for subjects taking lonafarnib. Coadministration with lonafarnib increased loperamide exposures and, therefore, the daily dose of loperamide is recommended not to exceed 1 mg for subjects taking lonafarnib. Coadministration with loperamide in this population has not been associated with cardiac dysrhythmias or respiratory depression, the treating physician should consider the risk:benefit if the patient continues to experience significant diarrhea on this dose. Any increases to the loperamide dose should be made gradually and the patient monitored for effects. Subjects generally tolerated lonafarnib and loperamide well. The majority of AEs were GI events that occurred during administration of lonafarnib, which were reduced in frequency following coadministration of lonafarnib and loperamide. The most common AEs were not unexpected based on the known safety profile of lonafarnib and loperamide.

### Example 3

A Phase 1, single-center, open-label, single-sequence, drug-drug interaction study to evaluate the effects of multiple-dose lonafarnib on the pharmacokinetics of single-dose omeprazole, a sensitive CYP2C19 substrate, and in parallel, a single-sequence, three-period crossover, pivotal food-effect evaluation with single-dose lonafarnib and a single-sequence, two-period crossover pivotal food effect evaluation with single-dose lonafarnib and ritonavir in healthy subjects.

To evaluate the effects of steady-state lonafarnib (LNF) on the pharmacokinetics (PK) of single-dose omeprazole in healthy subjects (Group 1).

To evaluate the effect of a high-fat/high-calorie standard breakfast on the single-dose PK of LNF relative to a fasted state in healthy subjects (Group 2).

To evaluate the effect of a low-fat/low-calorie breakfast on the single-dose PK of LNF relative to a fasted state in healthy subjects (Group 2).

This study was conducted as an open-label, drug-drug interaction (DDI) study, a food-effect study with LNF alone, and a food-effect study with LNF+ritonavir (RTV). This study assessed the effects of multiple-dose LNF (75 mg twice daily×5 days) on the PK of single dose omeprazole (1×40-mg capsule) relative to omeprazole alone in.

The effect of food (high-fat/high-calorie standard breakfast and low-fat/low-calorie breakfast) on the single-dose PK of LNF relative to the single-dose PK of LNF when administered under fasted conditions was evaluated.

CYP2C19 Direct Inhibition Evaluation and Steady-State Lonafarnib

Coadministration of single-dose omeprazole with multiple-dose LNF (75 mg twice daily for 5 consecutive days) resulted in moderate increases in single-dose omeprazole exposures.

Single-dose omeprazole Cₘₐₓ was increased 44% following coadministration with multiple-dose LNF and was observed at approximately the same time (Tₘₐₓ) with or without LNF.

The extent of single-dose omeprazole exposures (AUC₀₋ₜ and AUC_{0-inf}) were increased approximately 2-fold when omeprazole was coadministered with LNF.

Omeprazole CL/F and Kel following coadministration with LNF, were both decreased, while T1/2 was increased.

When omeprazole was coadministered with multiple-dose LNF, the omeprazole geometric mean ratio (GMR) and corresponding CI were 1.275 (1.01, 1.61), 1.602 (1.34, 1.91), and 1.600 (1.32, 1.94) for Cₘₐₓ, AUC₀₋ₜ, and AUC_{0-inf}, respectively.

These results show that steady-state LNF (75 mg twice daily for 5 days) increased single-dose omeprazole systemic exposures via direct/reversible inhibition of CYP2C19 in a moderate magnitude. Other sensitive CYP2C19 substrates should be monitored during concomitant administration with LNF. Multiple-dose PK parameters for LNF showed Cₘₐₓ (834 ng/mL) approximately 3 hours after dosing. Extent of exposure was 6,200 ng·h/mL and the T_{1/2} was 5.57 hours. Trough LNF concentrations increased from Day 7 to Day 9 (AM). By Day 10 trough levels stabilized, indicating steady-state LNF levels were achieved over 5 days of consecutive dosing. Omeprazole administered with and without multiple-dose LNF was generally well tolerated. There were no deaths, SAEs, or other significant AEs. All AEs were Grade 1 or 2. More subjects experienced AEs taking LNF than after taking omeprazole with or without LNF.

Food-effect evaluation: lonafarnib alone
Period 1: Oral dose of LNF (75-mg capsule×1) on Day 1 (AM) under fed conditions (high-fat/high-calorie breakfast)
Period 2: Oral dose of LNF (75-mg capsule×1) on Day 4 (AM) under fed conditions (low-fat/low-calorie breakfast)
Period 3: Oral dose of LNF (75-mg capsule×1) on Day 7 (AM) under fasted conditions

Administration of single-dose LNF with either a high-fat/high-calorie or a low-fat/low-calorie meal decreased the rate and extent of LNF absorption relative to a fasted state, and delayed Tₘₐₓ. Relative to the fasted state, the single-dose LNF Cₘₐₓ was decreased by 55% and 25% following a high-fat/high-calorie and low-fat/low-calorie breakfast, respectively.

The single-dose extent of LNF exposure (AUC₀₋ₜ and AUC_{0-inf}) was decreased 21-29%.

The CL/F was 33-35% higher in the 2 fed states compared with the fasted state.

When LNF was administered with a high-fat/high-calorie meal the GMR and corresponding CI were 0.471 (0.42, 0.53), 0.720 (0.65, 0.80), and 0.740 (0.67, 0.82) for Cₘₐₓ, AUC₀₋ₜ, and AUC_{0-inf}, respectively.

When LNF was administered with a low-fat/low-calorie meal the GMR and corresponding CI were 0.784 (0.66, 0.93), 0.835 (0.72, 0.97), and 0.833 (0.70, 0.99) for Cₘₐₓ, AUC₀₋ₜ, and AUC_{0-inf}, respectively.

These results confirm there is a food effect present when single-dose LNF is administered with a high-fat/high-calorie or a low-fat/low-calorie meal. A high-fat/high-calorie standard breakfast and a low-fat/low-calorie meal reduced single-dose exposures to LNF.

Although the foregoing invention has been described in some detail by way of illustration and example for purposes of clarity of understanding, one of skill in the art will appreciate that many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

All publications, patents, patent applications or other documents cited herein are hereby incorporated by reference in their entirety for all purposes to the same extent as if each individual publication, patent, patent application, or other document was individually indicated to be incorporated by reference for all purposes.

1. A method of treating a subject with laminopathies comprising: administering 150 mg/m² rounded to the nearest 25 mg/m² of lonafarnib to a subject, wherein the subject is further administered loperamide at a daily dose not to exceed 1 mg, and wherein the lonafarnib increases loperamide Cₘₐₓ by about 3-fold compared to loperamide administered alone.

2. The method of claim 1, wherein the subject is administered lonafarnib for a period of time of from between 12 months and 25 years or more.

3. The method of claim 1, wherein the subject is administered lonafarnib beginning at the age of 12 months.

4. The method of claim 1, wherein increases in dose of loperamide are made gradually.

5. The method of claim 4, wherein the dose is increased by 0.1 to about 0.2 mg.

6. (canceled)

7. The method of claim 1, wherein lonafarnib increases loperamide AUC₀₋ₜ by about 4-fold compared to loperamide administered alone.

8. A method of treating a subject with laminopathies comprising: administering 150 mg/m² rounded to the nearest 25 mg/m² of lonafarnib to a subject, wherein the sensitive substrates of CYP3A are contraindicated in subjects being administered lonafarnib.

9. The method of claim 8, wherein the subject is further administered midazolam and wherein there is about a 0.5-hour delay in Tₘₐₓ of midazolam when co-administered with lonafarnib.

10. The method of claim 9, wherein the significant increase in midazolam's systemic exposure are via a mechanism-based inhibition of cytochrome P450 CYP3A.

11. A method of treating a subject with laminopathies comprising: administering 150 mg/m² rounded to the nearest 25 mg/m² of lonafarnib to a subject, wherein lonafarnib is administered with food.

12. The method of claim 11, wherein the food is a high fat meal or a low-fat, low-calorie meal.

13. The method of claim 11, wherein food decreases lonafarnib Cₘₐₓ from between 25% to about 55% as compared to a fasted state.

14. The method of claim 11, wherein food decreases lonafarnib exposure (AUC₀₋ₜ and AUC_{0-inf}) from about 21% to about 29%.

15. The method of claim 11, wherein oral clearance (CL/F) is between about 33% to about 35% higher as compared to a fasted state when lonafarnib is administered with food.

16. The method of claim 11, wherein the Tₘₐₓ is delayed as compared to a fasted state.

17. The method of claim 1, wherein the subject is further administered omeprazole and wherein omeprazole Cₘₐₓ is increased about 44% following coadministration with lonafarnib.

18. The method of claim 17, wherein one or more of the omeprazole Cₘₐₓ following coadministration with LNF is at about the same as the Tₘₐₓ, or theomeprazole exposures (AUC₀₋ₜ and AUC_{0-inf}) were increased approximately 2-fold when omeprazole was coadministered with lonafarnib.

19. The method of claim 17, wherein omeprazole CL/F and Kei following coadministration with lonafarnib are decreased and the T_{1/2} is increased.

20. The method of claim 1, wherein CYP2C19 substrates are monitored during concomitant administration with lonafarnib.

21. The method of claim 1, wherein the lonafarnib is provided as 50 mg or 75 mg capsules.

22. The method of claim 1, wherein the laminopathies comprise one or more of Hutchinson-Gilford Progeria Syndrome and Progeroid Laminopathies.

23. The method of claim 1, wherein the laminopathies are associated with production of abnormally farnesylated lamin A proteins.

24. The method of claim 1, wherein one or more of the following is contraindicated with the use of lonafarnib: α1-adrenoreceptor antagonist, analgesics, antianginal, antineoplastic, antiarrhythmics, anti-gout, antimycobacterial, antipsychotics, antibiotic, antihistamines, ergot derivatives, GI motility agent, HMG Co-A reductase inhibitor, phosphodiesterase inhibitor, sedatives and hypnotics.

25. The method of claim 1, wherein one or more of the following is contraindicated with the use of lonafarnib: alfuzosin, propoxyphene, ranolazine, venetoclax, amiodarone, bepridil, dronedarone, quinidine, colchicine, rifabutin, lurasidone, clozapine, pimozide, quetiapine, fusidic acid, astemizole, terfenadine, dihydroergotamine, ergonovine, ergotamine, methylergonovine, cisapride, lovastatin, simvastatin, avanafil, sildenafil, vardenafil, clorazepate, diazepam, estazolam, flurazepam, midazolam and triazolam.

26. The method of claim 1, wherein concomitant use with strong or moderate CYP3A inhibitors or inducers is contraindicated.

27. The method of claim 1, wherein the use of lonafarnib in subjects with severe renal impairment is contraindicated.

28. A method of treating a subject with laminopathies comprising: administering 150 mg/m² rounded to the nearest 25 mg/m² of lonafarnib to a subject, wherein the subject is further administered loperamide at a daily dose not to exceed 1 mg, and wherein the lonafarnib increases loperamide AUC₀₋ₜ by about 4-fold compared to loperamide administered alone.
